(19) United States
(12) Patent Application Publication
(10) Pub. No.: US 2010/0297135 A1
(43) Pub. Date: Nov. 25, 2010

(54) EPITOPE FOR NEUTRALIZING ANTIBODIES

(75) Inventors: Charles Mackay, Vaucluse (AU); David Zahra, West Pennant Hills (AU)

Correspondence Address: HOWSON & HOWSON LLP, 501 OFFICE CENTER DRIVE, SUITE 210, FORT WASHINGTON, PA, 19034, US

(73) Assignee: CRC FOR ASTHMA AND AIRWAYS LTD., Sydney (AU)

(21) Appl. No.: 12/742,467

(22) PCT Filed: Nov. 12, 2008
(86) PCT No.: PCT/AU2008/001676

§ 371 Date: Jul. 20, 2010

Related U.S. Application Data
(60) Provisional application No. 60/987,254, filed on Nov. 12, 2007.

(51) Int. Cl. A61K 39/395; C07K 16/22; C07K 7/08; C07K 14/00; A61K 38/18; A61P 37/06; A61P 29/00; A61P 11/00; A61P 1/00; A61P 3/00

(52) U.S. Cl. 424/139.1; 530/387.9; 530/387.3; 530/324; 530/326; 514/7.6

(57) ABSTRACT

Provided herein are immunogenic peptides of GM-CSF comprising an amino acid sequence set forth in any one of SEQ ID NOs:2 to 6, or a portion thereof, and compounds and molecules that specifically interact with or bind to a region of GM-CSF located between, or corresponding to a region located between, about residues 89 and 117 of the human GM-CSF polypeptide sequence as set forth in SEQ NO:1.

### FIELD OF THE INVENTION

The present invention relates generally to peptides and peptide sequences derived from, or forming part of, granulocyte macrophage colony stimulating factor (GM-CSF). The peptides may be capable of eliciting an immune response and/or binding to anti GM-CSF antibodies. The invention also relates to compounds and molecules, such as antibodies, capable of recognising and interacting with the peptides and peptide sequences disclosed herein. The invention also relates to uses of such antigenic peptides and compounds or molecules, such as monoclonal antibodies, capable of binding thereto.

### BACKGROUND OF THE INVENTION

Granulocyte-macrophage colony stimulating factor (GM-CSF) is a hematopoietic growth factor which regulates the differentiation, proliferation and function of granulocytes and monocytic cells such as macrophages.

GM-CSF is also a potent inflammatory cytokine, the activity or overexpression of which can have significant detrimental effects. GM-CSF is implicated in a variety of autoimmune and inflammatory diseases including rheumatoid arthritis, asthma, multiple sclerosis and idiopathic thrombocytopenic purpura. In asthma and rheumatoid arthritis, elevated levels of GM-CSF have been detected and correlated with the inflammatory process, whilst in experimental autoimmune encephalomyelitis, an animal model of multiple sclerosis, GM-CSF knockout mice are protected against the onset of the disease.

There is a clear need for the development of effective approaches to target GM-CSF and block or neutralize GM-CSF activity. Antibodies against GM-CSF offer one particularly suitable alternative as antagonists of GM-CSF with clear therapeutic applications, such as in the treatment or prevention of autoimmune or inflammatory conditions.

Thus, there is also a need for suitable methods for inducing a neutralizing immune response against GM-CSF, and for generating and isolating or purifying anti-human GM-CSF antibodies.

### SUMMARY OF THE INVENTION

The present invention is predicated on the inventors' determination of a region on the surface of the human granulocyte macrophage colony stimulating factor (GM-CSF) molecule that comprises an epitope recognised and targeted by antibodies capable of neutralizing the activity of human GM-CSF.

According to one aspect, the present invention provides a compound or molecule that specifically binds to a region of GM-CSF located between, or corresponding to a region located between, about residues 89 and 117 of the human GM-CSF polypeptide sequence as set forth in SEQ ID NO:1.

In a particular embodiment, the molecule is an antibody. The region of human GM-GSF bound by the antibody may comprise or consist of an amino acid sequence set forth in any one of SEQ ID NOs:2 to 6, or a portion thereof. The GM-CSF may be a primate GM-CSF, typically human GM-CSF, or a recombinant GM-CSF comprising a sequence set forth in any one of SEQ ID NOs:2 to 6, or a portion thereof.

In an embodiment the region of human GM-GSF bound by the antibody comprises the amino acid sequence set forth in SEQ ID NO:6.

The antibody may be a neutralizing antibody capable of inhibiting the expression and/or activity of GM-CSF thereby inhibiting one or more biological functions of GM-CSF. The antibody may be a polyclonal or monoclonal antibody. By way of example only, the monoclonal antibody may be the antibody designated mAb 4K21, or a humanized or human form thereof.

In a further aspect, the present invention provides an immunogenic peptide comprising an amino acid sequence set forth in any one of SEQ ID NOs:2 to 6, or a portion thereof.

In a further aspect, the present invention provides an immunogenic peptide consisting of an amino acid sequence set forth in any one of SEQ ID NOs:2 to 6, or a portion thereof.

According to the above aspects the immunogenic peptide may be a constituent of, or be derived from, human GM-CSF having the amino acid sequence set forth in SEQ ID NO:1. Typically, the immunogenic peptide binds to a polyclonal or monoclonal antibody obtained in response to immunising a mammal with human GM-CSF or a fragment, variant or derivative thereof.

According to a further aspect, the present invention provides an isolated polynucleotide encoding an immunogenic peptide of the invention.

According to a further aspect, the present invention provides a compound or molecule that binds to an immunogenic peptide of the invention.

Also provided by the present invention is the use of an immunogenic peptide as disclosed herein for the generation, isolation and/or detection of a compound or molecule capable of interacting with GM-CSF.

Also provided by the present invention is the use of an immunogenic peptide as disclosed herein for the generation, isolation and/or detection of a compound or molecule capable of interacting with GM-CSF. In an embodiment the molecule is an antibody. In a particular embodiment, the antibody specifically binds to human GM-CSF. The antibody may be a neutralizing antibody.

A further aspect of the invention provides the use of an immunogenic peptide of the invention for eliciting an immune response in a mammal.

In a further aspect the present invention provides a method for the treatment or prevention of a GM-CSF-mediated disease or condition or a disease or condition otherwise associated with elevated or aberrant GM-CSF expression and/or activity, the method comprising administering to a subject in need thereof an effective amount of an immunogenic peptide of the invention to thereby generate an immune response against GM-CSF.

In a further aspect the present invention provides a method for the treatment or prevention of a GM-CSF-mediated disease or condition or a disease or condition otherwise associated with elevated or aberrant GM-CSF expression and/or activity, the method comprising administering to a subject in need thereof an effective amount of a compound or molecule that binds to region of human GM-CSF located between about residues 89 and 117 of the human GM-CSF polypeptide sequence as set forth in SEQ ID NO:1 and/or an immunogenic peptide of the invention.

In accordance with the above methods, typically the disease or condition is an autoimmune or inflammatory disease or condition. The disease or condition may be selected from, for example, asthma, rheumatoid arthritis, chronic obstructive pulmonary disease, idiopathic thrombocytopenic purpura, acute respiratory distress syndrome, multiple sclerosis, Alzheimer's disease, Crohn's disease, irritable bowel syndrome, colitis, psoriasis, macular degeneration, uveitis, Wallerian degeneration, antiphospholipid syndrome, restinosis, atherosclerosis, idiopathic pulmonary fibrosis, relapsing polychondritis, hepatitis, glomerulonephritis, lupus and other metabolic diseases.

In a further aspect the present invention provides the use of an immunogenic peptide or compound or molecule, such as an antibody, of the invention in the manufacture of a medicament for treating or preventing a GM-CSF-mediated disease or condition or a disease or condition otherwise associated with elevated or aberrant GM-CSF expression and/or activity.

In another aspect, the present invention provides the use of an immunogenic peptide or compound or molecule, such as an antibody, of the invention for use in the treatment or prevention of a GM-CSF-mediated disease or condition or a disease or condition otherwise associated with elevated or aberrant GM-CSF expression and/or activity.

The invention further provides pharmaceutical compositions comprising one or more immunogenic peptides or compounds or molecules, such as antibodies, of the invention, optionally together with suitable pharmaceutically acceptable carriers and/or diluents.

### BRIEF DESCRIPTION OF THE DRAWINGS

The present invention will now be described, by way of non-limiting example only, with reference to the accompanying drawings.

FIG. 1. An alignment of polypeptide sequences of human GM-CSF, murine GM-CSF and mutants M1 to M5. Conserved residues are shaded. Boxed residues in the mutant sequences denote those residues altered from the wild type human sequence to reflect corresponding murine residues.

FIG. 2. Binding analysis of 4K21 mAb to various GM-CSF polypeptides as determined by semi-quantitative ELISA. WT (wild type human GM-CSF) and mutants M1 to M5b are bacterially expressed GM-CSF polypeptides. The ability of 4K21 mAb (grey lines) to bind GM-CSF polypeptides was compared to that of a commercially available anti human GM-CSF antibody (BD) (black lines).

FIG. 3. Determination of the binding epitope of 4K21 mAb by Western blot analysis. The relative expression of GM-CSF polypeptides were assessed semi-quantitatively on a Coomassie stained gel. Total protein amounts were estimated based on the Coomassie gel. The following indicated amounts of total polypeptide were separated by PAGE, transferred to nitrocellulose and probed with 4K21 antibody: 1, wild type human GM-CSF (150 ng); 2, pET (5 μg); 3, mutant M1 (300 ng); 4, mutant M2 (1 μg); 5, mutant M3 (5 μg); 6, mutant M4 (1 μm); 7, mutant M5 (5 μg); and 8, wild type Macaque GM-CSF (5 μg).

FIG. 4. Binding analysis of antibodies to murine GM-CSF mutant polypeptides comprising the human-derived mutant regions M3, M4, M5 or M5b as determined by semi-quantitative ELISA. In the case of murine GM-CSF comprising hM5b, the binding of 4K21 mAb (dark grey) was compared to that of a rat anti-mouse GM-CSF antibody (BD) (light grey). For murine GM-CSF comprising hM3, hM4 or hM5 the binding of a humanized form of 4K21 (hGM4/34) (dark grey) was compared to that of the BD rat anti-mouse GM-CSF antibody (light grey).

FIG. 5. Binding analysis of 4K21 mAb (grey) and BD anti-human GM-CSF antibody (black) to site-directed (alanine) mutants of human GM-CSF. Alanine mutants are designated by the location of the mutation in the human GM-CSF sequence as depicted in SEQ ID NO:1.

FIG. 6. Representations of the binding epitope of 4K21 mAb on three-dimensional modelled structure of human GM-CSF using Pymol protein display software. Two different orientations of the GM-CSF molecule are shown (A and B) with the M5b region highlighted.

Amino acid and nucleotide sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided at the end of the specification. Specifically, the amino acid sequence of wild type human GM-CSF is provided in SEQ ID NO:1. Amino acid sequences of the regions of human GM-CSF containing a neutralizing epitope as disclosed herein are provided in SEQ ID NOs:2 to 6. The amino acid sequences of variable light chain and variable heavy chain of the exemplary monoclonal antibody 4K21 are set forth in SEQ ID NOs:7 and 8. The sequences of oligonucleotide primers used in the construction of mutant GM-CSF constructs as described herein are set forth in SEQ ID NOs:9 to 38.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “activity” as it relates to GM-CSF means any cellular function, action, effect or influence exerted by the GM-CSF, either by the protein or polypeptide itself or any fragment or portion thereof.

In the context of the present specification reference to an “antibody” or “antibodies” includes reference to all the various forms of antibodies, including but not limited to whole antibodies, antibody fragments, including, for example, Fv, Fab, Fab′ and F(ab′)₂ fragments, domain antibodies, humanized antibodies, human antibodies, nanobodies and immunoglobulin-derived polypeptides produced through genetic engineering techniques.

In the context of the present specification reference to “binding” of an antibody means binding, interacting or associating with or to a target antigen such as GM-CSF. Reference to “GM-CSF” includes fragments or portions thereof which comprise the epitope(s) to which an antibody binds. Consequently, reference to an antibody binding to GM-CSF includes within its scope the binding, interaction or association of the antibody or an antigen-binding portion thereof to part, fragment or epitope-containing region of GM-CSF. Generally, “binding”, “interaction” or “association” means or includes the specific binding, interaction or association of the antibody to GM-CSF or a portion thereof. In the broader context of a compound or molecule that “interacts” with GM-CSF, such interaction may be direct or indirect.

As used herein the term “effective amount” includes within its meaning a non-toxic but sufficient amount of an agent to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein in the context of peptides of the invention the term “immunogenic” means a peptide that is either capable of inducing an immune response in an organism (either when administered alone or together with a suitable adjuvant or carrier molecule) or is capable of being recognised by and binding to an antibody. Thus the term “immunogenic” is used in its broadest context and does not require that the peptide be capable of itself inducing an immune response upon administration but that the peptide be capable of interacting with a component of the immune response, namely an antibody.

The terms “inhibits” and “inhibiting” as used herein as they relate to the expression or activity of GM-CSF does not necessarily mean completely inhibiting expression or activity. Rather, expression or activity may be inhibited to an extent, and/or for a time, sufficient to produce the desired effect. Thus inhibition of GM-CSF expression or activity may be partial or complete attenuation of the biological effect(s) of GM-CSF and such inhibition may be temporally limited.

The term “neutralizing” as used herein with reference to an antibody means an antibody capable of inhibiting, partially or completely, the activity of GM-CSF. The inhibition of activity of GM-CSF may be determined by an inhibition in at least one biological function of GM-CSF which in turn may, in some circumstances, be detected by the amelioration, removal or reduction in one or more symptoms of a GM-CSF-mediated disease or condition or a disease or condition associated with elevated or aberrant GM-CSF expression and/or activity. In the context of an epitope, the term “neutralizing” refers to an epitope to which a neutralizing antibody binds.

The term “polypeptide” means a polymer made up of amino acids linked together by peptide bonds. The terms “polypeptide” and “protein” are used interchangeably herein, although for the purposes of the present invention a “polypeptide” may constitute a portion of a full length protein.

The term “subject” as used herein typically refers to mammals including humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As disclosed herein, the inventors have elucidated the sequence of a neutralizing epitope located on the surface of the human GM-CSF molecule. This opens up numerous new avenues for the identification, purification and detection of novel compounds and molecules, such as antibodies, aptamers, or small molecule inhibitors directed against GM-CSF and thus offers the potential for new therapeutic treatment for diseases and conditions mediated by GM-CSF or associated with elevated or aberrant levels of GM-CSF expression and/or activity. The identification of a novel GM-CSF epitope also enables the production of novel immunogenic peptides, and polynucleotides encoding the same, and the use of such peptides and polynucleotides in eliciting an immune response against GM-CSF.

While exemplified herein in relation to human GM-CSF polypeptide sequences, those skilled in the art will readily appreciate that the invention is not limited to applications pertaining to human GM-CSF. For example, as exemplified herein, the macaque GM-CSF protein shows high amino acid sequence similarity to human GM-CSF and binds the exemplary monoclonal antibody 4K21. Thus, the present invention relates to any GM-CSF polypeptide comprising an amino acid sequence as set forth in any one of SEQ ID NOs:2 to 6, a portion thereof or a substantially identical sequence. By “substantially identical” sequence is meant a sequence comprising one or more amino acid substitutions, deletions or insertions when compared with a sequence of SEQ ID NOs:2 to 6, which alterations do not substantially impair the ability of the epitope sequence to recognise an antibody and generate an immune response under appropriate conditions. Thus, not only is human GM-CSF contemplated by the present invention, but also, for example other primate GM-CSF sequences and any recombinant GM-CSF polypeptide or GM-CSF-derived peptide or polypeptide comprising an amino acid sequence as set forth in any one of SEQ ID NOs:2 to 6, a portion thereof or sequence substantially identical thereto.

It will also be appreciated that the present invention encompasses GM-CSF epitopes, immunogenic peptides derived therefrom and compounds or molecules recognising such epitopes and/or immunogenic peptides, wherein the epitope or immunogenic peptide comprises one or more additional amino acid residues flanking a sequence as set forth in any one of SEQ ID NOs:2 to 6, or portion thereof. Such additional residues may be derived from GM-CSF or any other polypeptide. By “portion” is meant an amino acid sequence that comprises at least a segment or portion of an amino acid sequence set forth in any one of SEQ ID NOs:2 to 6, wherein the portion retains immunogenicity. The immunogenicity of the portion need not be identical to that of the larger sequence of SEQ ID NOs:2 to 6 from which it is derived, but may be reduced or enhanced in comparison. The “portion” may comprise one or more additional amino acid residues flanking the sequence derived from SEQ ID NOs:2 to 6, which additional residues may be derived from GM-CSF or any other polypeptide.

The epitope as disclosed herein and immunogenic peptides comprising the epitope may be targeted by any compound or molecule that is capable of interacting with the epitope directly or indirectly. The compound or molecule may be, for example, an antibody, a small molecule inhibitor, or a peptide or nucleic acid aptamer. In one embodiment, the molecule is an antibody or a portion or derivative thereof as defined herein. The disclosure following is provided in the context of antibodies by way of exemplification only. Those skilled in the art will appreciate that the invention is not so limited, and that any suitable interacting compound or molecule is contemplated. Accordingly, the invention contemplates methods for the identification of compounds or molecules capable of interacting with the region of GM-CSF located between about residues 89 and 117 of the human GM-CSF polypeptide sequence as set forth in SEQ ID NO:1, the methods comprising contacting a candidate compound or molecule with GM-CSF or a portion or fragment thereof comprising said region and assaying for said interaction. The interaction may be binding, and the assay may involve assaying for the formation of a complex between the candidate compound or molecule and the GM-CSF or portion or fragment thereof.

According to one aspect, the present invention provides an antibody that specifically binds to a region of human GM-CSF located between about residues 89 and 117 of the human GM-CSF polypeptide sequence as set forth in SEQ ID NO:1. The region of human GM-GSF bound by the antibody may comprise or consist of an amino acid sequence set forth in any one of SEQ ID NOs:2 to 6, or a portion thereof. In an embodiment the region bound by the antibody comprises the amino acid sequence set forth in SEQ ID NO:6.

Suitable antibodies may be polyclonal or monoclonal. Also contemplated are antigen-binding fragments of polyclonal and monoclonal antibodies. The antibodies may be humanized or human antibodies suitable for administration to humans. These include humanized antibodies prepared, for example, from murine monoclonal antibodies and human monoclonal antibodies which may be prepared, for example, using transgenic mice or by phage display.

Antibodies may be prepared by a variety of procedures well known to those skilled in the art. For example, reference may be had to Monoclonal Antibodies, Hybridomas: A New Dimension in Biological Analyses, Kennet et al. (eds.), Plenum Press, New York (1980); Antibodies: A Laboratory Manual, Harlow and Land (eds.), Cold Spring Harbor Laboratory Press, Cold Spring Harbor, N.Y., (1988); and Monoclonal Antibodies: Principles and Practice, Goding, 3rd Edition, Academic Press (1996). The disclosures thereof are incorporated herein by reference in their entirety. Similarly, monoclonal antibodies secreted by hybridoma cell lines may be purified by conventional techniques.

By way of example, one method for producing an antibody of the present invention comprises immunizing a non-human animal, such as a mouse or a transgenic mouse, with a GM-CSF polypeptide, or a fragment thereof comprising an amino acid sequence as set forth in any one of SEQ ID NOs:2 to 6, whereby antibodies directed against the GM-CSF polypeptide are generated in said animal. The GM-CSF polypeptide or fragment thereof may be from any mammalian source. Typically, the GM-CSF polypeptide or immunogenic portion of fragment thereof is human GM-CSF.

Examples of monoclonal antibodies that bind to the GM-CSF epitope identified herein are murine monoclonal antibody 4K21 and human and humanized forms thereof. Murine monoclonal antibody 4K21 comprises the variable light chain sequence as set forth in SEQ ID NO:3 and the variable heavy chain sequence as set forth in SEQ ID NO:4. A hybridoma producing murine monoclonal antibody 4K21 was deposited on 17 May 2007 at the European Collection of Cell Cultures (ECACC), Centre for Applied Microbiology and Research, Porton Down, Salisbury, United Kingdom, under Accession No. 07051601. Monoclonal antibody 4K21 is described in more detail in co-pending application no. PCT/AU2008/000728, the disclosure of which is incorporated herein in its entirety.

Antigen-binding fragments of antibodies may be produced by conventional techniques. Examples of such fragments include, but are not limited to, Fab, Fab′, F(ab′) 2 and Fv fragments, including single chain Fv fragments (termed sFv or scFv). Antibody fragments and derivatives produced by genetic engineering techniques, such as disulphide stabilized Fv fragments (dsFv), single chain variable region domain (dAbs) molecules, nanobodies and minibodies are also contemplated for use. Unless otherwise specified, the terms “antibody” and “monoclonal antibody” as used herein encompass both whole antibodies and antigen-binding fragments thereof.

Such derivatives of monoclonal antibodies directed against GM-CSF may be prepared and screened for desired properties, by known techniques. The techniques may involve, for example, isolating DNA encoding a polypeptide chain (or a portion thereof) of a monoclonal antibody of interest, and manipulating the DNA through recombinant DNA technology. The DNA may be used to generate another DNA of interest, or altered (e.g. by mutagenesis or other conventional techniques) to add, delete, or substitute one or more amino acid residues, for example. DNA encoding antibody polypeptides (e.g. heavy or light chain, variable region only or full length) may be isolated from B-cells of mice that have been immunized with GM-CSF. The DNA may be isolated by conventional procedures including polymerase chain reaction (PCR).

Phage display is an alternative example of a suitable technique whereby derivatives of antibodies of the invention may be prepared. In one approach, polypeptides that are components of an antibody of interest are expressed in any suitable recombinant expression system, and the expressed polypeptides are allowed to assemble to form antibody molecules.

Single chain antibodies may be formed by linking heavy and light chain variable region (Fv region) fragments via an amino acid bridge (short peptide linker), resulting in a single polypeptide chain. Such single-chain Fvs (scFvs) may be prepared by fusing DNA encoding a peptide linker between DNAs encoding the two variable region polypeptides (VL and VH). The resulting antibody fragments can form dimers or trimers, depending on the length of a flexible linker between the two variable domains (see Kortt et al., Protein Engineering 10: 423, 1997). Techniques developed for the production of single chain antibodies include those described in U.S. Pat. No. 4,946,778; Bird (Science 242: 423, 1988), Huston et al. (Proc. Natl. Acad Sci USA 85: 5879, 1988) and Ward et al. (Nature 334: 544, 1989). The disclosures thereof are incorporated herein by reference in their entirety. Single chain antibodies derived from antibodies provided herein are encompassed by the present invention.

For therapeutic and diagnostic purposes antibodies are typically generated in and isolated from non-human animals, such as mice and rabbits. However antibodies derived from non-human animals are generally unsuitable for administration to humans as they may cause an immune response and result in the generation of anti-mouse antibodies (the so-called HAMA response). The HAMA response can neutralize the mouse antibodies by rapidly clearing them from the blood, thus preventing the mouse antibody from binding to its target.

To avoid development of a HAMA response one strategy is to “humanize” the mouse antibody by replacing as many “foreign” residues in the non-epitope binding regions with human sequences. The specificity of the interaction between an antibody and an antigen involves the hypervariable or complementarity-determining regions (CDRs) in the variable domain. These residues are generally not changed during the humanization process. The remaining residues in the variable domain, referred to as the framework (FW) and the constant regions of the antibody, on both heavy and light chains are usually replaced with human sequences. To avoid disrupting the structure of the antibody-binding pocket, and the specificity or affinity of the antibody, certain mouse residues in the framework regions may need to be preserved. Suitable humanization processes, such as CDR grafting, are well known to those skilled in the art. A particularly suitable approach is exemplified herein. Procedures for the production of chimeric and humanized monoclonal antibodies also include those described in, for example, Riechmann et al., Nature 332: 323, 1988, Liu et al., Proc. Natl. Acad. Sci. USA 84: 3439, 1987, Larrick et al., Bio/Technology 7: 934, 1989 and Winter and Harris, TIPS 14: 139, 1993. The complementarity determining regions (CDRs) of a given antibody may be identified using the system described by Kabat et al. in Sequences of Proteins of Immunological Interest, 5th Ed., US Dept. of Health and Human Services, PHS, NIH, NIH Publication No. 91-3242, 1991).

As an exemplary alternative, aptamers may be designed to recognise a human GM-CSF epitope as disclosed herein. Suitable aptamers may be designed using techniques known to those skilled in the art and using any suitable chemical scaffold. For example, aptamers may be nucleic acid-based (DNA or RNA, or modifications thereof) or peptide-based. Similarly, selection of specific aptamers may be achieved by methods known to those skilled in the art such as, for example, systematic evolution of ligands by exponential enrichment (SELEX) in the case of nucleic acid aptamers and the yeast two cell hybrid system for peptide aptamers.

Also provided by the present invention are immunogenic peptides derived from human GM-CSF, such peptides comprising or consisting of an amino acid sequence set forth in any one of SEQ ID NOs:2 to 6, or a portion thereof. Further provided are uses of such peptides for, inter alia, eliciting an immune response, generating antibodies, isolating or purifying antibodies, detecting antibodies and preventing or treating GM-CSF-mediated diseases or conditions or diseases or conditions otherwise associated with elevated or aberrant GM-CSF expression and/or activity.

Immunogenic peptides of the invention may be used to evoke an immune response upon administration to a suitable subject. Accordingly, such immunogenic peptides find application in the generation of neutralizing antibodies, for example monoclonal antibodies, in a suitable host animal, which antibodies can be isolated or purified by techniques well known to those skilled in the art and used for therapeutic or prophylactic purposes as described herein. That is, immunogenic peptides of the invention may be used to immunise a suitable non-human animal for the production of monoclonal antibodies by known techniques such as those hereinbefore mentioned.

Further, immunogenic peptides of the invention may be administered to a subject directly for the purpose of achieving a prophylactic or therapeutic outcome by virtue of the induction of an immune response by the peptides. Thus, immunogenic peptides may be formulated into suitable pharmaceutical compositions.

For the purposes of generating an immune response, whether for the purposes of the generation of antibodies in an animal model or for direct therapeutic or prophylactic application, immunogenic peptides of the invention may be administered together with one or more antigenic adjuvants to promote or enhance immunogenicity. Suitable adjuvants include but are not limited to Complete Freund's Adjuvant (CFA) and aluminium hydroxide. Also suitably, the immunogenic peptides may be conjugated with suitable carrier molecules to enhance immunogenicity. A variety of suitable carrier molecules or adjuvants are known to those skilled in the art, including for example, bovine serum albumin (BSA), human serum albumin (HSA), keyhole limpet haemocyanin (KLH), edestin, thyroglobulin, erythrocytes such as sheep erythrocytes (SRBC), anatoxins, and polyamino acids such as, poly D-lysine or poly D-glutamic acid. Methods for the conjugation of peptides with carrier molecules to promote or enhance the production of an immune response are well known to those skilled in the art.

Immunogenic peptides of the invention also find application in the isolation or purification from a biological sample of antibodies, polyclonal or monoclonal; which antibodies are generated against GM-CSF. The sample may be any suitable sample, including cell culture fluid, or blood sample, serum sample or a sample of other bodily fluid or tissue. By way of example, the antibodies may be purified by any suitable purification technique known to those skilled in the art such as affinity purification where a peptide of the invention is used as a purification reagent. For affinity purification the peptide may be immobilised on a solid support such as a column, magnetic beads, glass beads, or in a matrix such as silica gel, agarose or polyacrylamide and the sample containing, or putatively containing anti GM-CSF antibodies contacted with the solid support under conditions such that binding between the immobilised peptides and the antibodies can take place.

Immunogenic peptides of the invention may also be used to detect the presence of antibodies against GM-CSF in a sample. Assays for the detection of such antibodies may find application, for example, in monitoring the progress and efficacy of therapies for the treatment of GM-CSF-mediated diseases or conditions, diseases or conditions otherwise associated with elevated levels of expression and/or activity of GM-CSF. Detection assays may also be employed in a ‘quality control’ role, for example in evaluating the quality and/or integrity of a commercially produced batch of antibodies.

The detection assay may be any suitable immunological assay, such as an enzyme-linked immunosorbent assay (ELISA), a radioimmunological assay, a fluorescence-based immunological assay, a Western blot, or any other technique in which the antibody or antigen is marked with a detectable molecule or with any other indicator means. Methods for the detection of antibodies are well known to those skilled in the art.

The present invention provides methods for treating or preventing GM-CSF-mediated diseases or conditions, diseases or conditions otherwise associated with elevated levels of expression and/or activity of GM-CSF, and other diseases or conditions which may be beneficially treated by inhibiting or neutralizing GM-CSF activity. Diseases and conditions which may be treated in accordance with the present invention include autoimmune and inflammatory diseases. Such diseases include but are not limited to asthma, rheumatoid arthritis, chronic obstructive pulmonary disease, idiopathic thrombocytopenic purpura, acute respiratory distress syndrome, multiple sclerosis, Alzheimer's disease, Crohn's disease, irritable bowel syndrome, colitis, psoriasis, macular degeneration, uveitis, Wallerian degeneration, antiphospholipid syndrome, restinosis, atherosclerosis, idiopathic pulmonary fibrosis, relapsing polychondritis, hepatitis, glomerulonephritis, lupus and other metabolic diseases. Additional autoimmune diseases which may be treated in accordance with the invention include systemic sclerosis, scleroderma, Sjogren syndrome, spondyloarthritis, Sapho syndrome, juvenile iodipathic arthritis, lyme disease, polymyositis, dermatomyositis, autoimmune thyroditis, Grave's disease, Type 1 diabetes, adrenaltis, autoimmune Addison's disease, polyendocrine syndromes, gastritis, pernicious anemia, hypophysitis, hemolytic anemia, neutropenia, aplastic anemia, clotting disorder including acquired von Willebrand syndrome, Guillain-Barre Syndrome, chronic inflammatory demyelinating polyradiculoneuropathy, myasthenia gravis, Lambert-Eaton myasthenic syndrome, acquired neuromyotonia, Stiff-Person Syndrome, Cerebellar Ataxia, Rasmussen Encephalitis, Morvan Syndrome, Limbic encephalitis, ocular disease, inner ear disease, celiac disease, primary billary cirrhosis, primary sclerosing cholangitis, pancreatitis, pemphigus pemphigoid, alopecia areata, vitiligo, chronic urticaria, Goodpasture's disease, ANCA-associated glomerulonephritis, orchitis, oophoritis, rheumatic heart disease, myocarditis, dilated cardiomyopathy, polyartheritis nodosa, Kawasaki's disease, Wegener's granulomatosis, microscopic polyangiitis, Churg-Strauss syndrome, cryoglobulenemic vasculitis, Henoch-Schonlein purpura, Behcet's disease, giant cell arteritis, Takayasu's arteritis, idiopathic bronchiolitis obliterans, idiopathic pulmonary fibrosis, autoimmune disorder of the lung and Opsoclonus-Myoclonus syndrome.

Immunogenic peptides and antibodies disclosed herein also find application in the treatment of failed or rejected implants and prostheses and failed or rejected organ transplants, such as for example lung, kidney, heart and liver.

Additional applications, both in vivo and in vitro, of immunogenic peptides and antibodies of the invention are contemplated. For example, antibodies of the invention may be employed in assays designed to detect the presence of GM-CSF and/or to purify GM-CSF.

For therapeutic and prophylactic applications, immunogenic peptides or antibodies of the invention are administered to a subject in need thereof in an amount effective to obtain the desired therapeutic or prophylactic effect. It will be understood that the specific effective amount or dose for any particular subject will depend upon a variety of factors including, for example, the activity of the specific molecule(s) employed, the age, body weight, general health and diet of the individual to be treated, the time of administration, rate of excretion, and combination with any other treatment or therapy. Single or multiple administrations can be carried out with dose levels and pattern being selected by the treating physician.

In treating or preventing autoimmune and inflammatory conditions, the present invention contemplates the administration of multiple peptides or multiple antibodies if required or desirable. Whether it is suitable or desirable to administer one or more peptides or antibodies can be determined by those skilled in the art on a case-by-case basis.

The invention also contemplates combination therapies, wherein peptides or antibodies as described herein are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours, days, weeks, months or years between the administration of the two agents. These agents may be administered in any order.

According to embodiments of the invention, peptides and antibodies may be administered in any suitable form. In accordance with the present invention such molecules are typically administered in the form of pharmaceutical compositions, which compositions may comprise one or more pharmaceutically acceptable carriers, excipients or diluents. Such compositions may be administered systemically, regionally or locally and via any suitable route such as by parenteral (including intravenous, intraarterial or intramuscular), oral, nasal, topical and subcutaneous routes.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration. Adjuvants typically include emollients, emulsifiers, thickening agents, preservatives, bactericides and buffering agents.

For administration as an injectable solution or suspension, non-toxic parenterally acceptable diluents or carriers can include, Ringer's solution, medium chain triglyceride (MCT), isotonic saline, phosphate buffered saline, ethanol and 1,2 propylene glycol. Methods for preparing parenterally administrable compositions are known to those skilled in the art, and are described in more detail in, for example, Remington's Pharmaceutical Science, 15th ed., Mack Publishing Company, Easton, Pa., hereby incorporated by reference in its entirety.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Murine Anti-Human GM-CSF Monoclonal Antibody 4K21

Anti-human GM-CSF antibodies were generated by intraperitoneal injection of 50 μg of recombinant human GM-CSF (Peprotech) in CFA (complete Freund's adjuvant) into BALB/c mice. This was followed by two further injections of 25 μg of recombinant human GM-CSF in IFA (incomplete Freund's adjuvant) 2 weeks and 4 weeks later.

Six months after the initial injection, a 25 μg booster of recombinant human GM-CSF in IFA was administered intraperitoneally. Finally 2 weeks after the booster 50 μg of recombinant human GM-CSF in phosphate buffered saline was administered intravenously. Five days later mice spleens were harvested and fused with SP2/0 cells.

Antibody expressing hybridoma supernatants were screened by ELISA (enzyme linked immunosorbent assay) for specific binding to recombinant human GM-CSF. One murine anti-human GM-CSF monoclonal antibody generated was designated 4K21-O11-E14 (hereinafter ‘4K21 mAb’).

The variable region amino acid sequences of 4K21 mAb are as follows:

Light chain
(SEQ ID NO: 7)
DVVMTQTPLSLPVSLGDQASISCRSSQSLVNSNGNTYLHWFLQKPGQSP
KLLIYKVSNRFSGVPDRFSGSGSGTDFTLKISRVEAEDLGVYFCSQSTH
VPPTFGGGTKLEIK
Heavy chain
(SEQ ID NO: 8)
EVQLVESGGGLVKSGGSLKLSCAASGFAFSAYDMSWVRQTPEKRLELVA
YISSGGSSFYYPDTVKGRFTISRDNAKNTLYLQMSSLKSEDTAMYYCTR
HLGFDYWGQGTTLTVSS

The hybridoma containing 4K21 was deposited on 17 May 2007 at the European Collection of Cell Cultures (ECACC), Centre for Applied Microbiology and Research, Porton Down, Salisbury, United Kingdom, under Accession No. 07051601.

The 4K21 mAb was demonstrated to have a high binding affinity for human GM-CSF. The binding kinetics were determined using a Biacore and compared with the binding kinetics for a commercially available rat anti-human GM-CSF antibody from BD Biosciences (BD; Cat No. 554501) (see Table 1 below). Recombinant human GM-CSF was coated onto a CM5 chip to determine the binding kinetics of 4K21 mAb. An uncoated flow cell was used as a reference. The antibody, prepared in HBS-EP at concentrations of 66.7 nM to 4.17 nM with 2-fold serial dilution, was injected for 2 minutes with a stabilisation time of 10 minutes and dissociation time of 15 minutes. The BIACore kinetic analysis wizard was run to determine each antibody's association (ka [1/Ms]), dissociation (kd[1/s]) and affinity (KD [nM]) to GM-CSF. As shown in Table 1, 4K21 mAb displays an extremely high binding affinity for human GM-CSF, binding at low picomolar concentrations (approximately 200 pM). Using an alternative method of antibody capture (Biacore), the binding affinity of 4K21 mAb to human GM-CSF was determined to be approximately 430 pM. Moreover, binding of 4K21 mAb to human GM-CSF is highly specific. The antibody does not bind murine GM-CSF, human G-CSF or human IL-21.

TABLE 1
Binding kinetics of 4K21 mAb.

| Antibody | ka (1/Ms) | kd (1/s) | KA (1/M) | KD (M) | KD (nm) |
|---|---|---|---|---|---|
| BD | 5.20E+04 | <1e−5 | >5.2e9 | <1.9e−10 | <0.19 |
| 4K21 mAb | 5.90E+04 | <1e−5 | >5.9e9 | <1.7e−10 | <0.17 |

Recombinant human GM-CSF was coated onto a CM5 chip to determine the binding kinetics of 4K21 mAb.

It has also been shown that 4K21 mAb is an efficient neutralizer of human GM-CSF activity. 4K21 mAb was demonstrated to be able to neutralize the growth promoting function of human GM-CSF in a TF-1 cell growth bioassay. TF-1 cells are dependant on the presence of GM-CSF for their growth and are maintained with 2 ng/ml recombinant GM-CSF (Peprotech). A range of antibody concentrations (10 ng/ml to 10,000 ng/ml) was prepared in TF-1 growth media with 0.25 ng/ml human GM-CSF. 4K21 mAb and GM-CSF were mixed at each concentration and incubated for 1 hr at 37° C. in a 96 well plate. Washed TF-1 cells (1×10⁵) were then added to each well and incubated at 37° C. for 72 hrs. Cellular growth was quantitated by pulsing each well for 4 hrs with 0.5 uCi of ³H-thymidine. From 13 independent experiments, the average IC₅₀ was determined to be approximately 308 pM. In contrast, in similar experiments conducted using murine GM-CSF, 4K21 mAb was unable to neutralize murine GM-CSF mediated growth of FDC-P1 cells (data not shown), thereby demonstrating the anti-human GM-CSF specificity of 4K21.

The production of monoclonal antibody 4K21 is described in more detail in co-pending application no. PCT/AU2008/000728, the disclosure of which is incorporated herein in its entirety.

### Example 2

### Mutant GM-CSF Molecules

In order to elucidate the neutralizing epitope of human GM-CSF to which the exemplary neutralizing antibody 4K21 mAb binds, a series of recombinant mutant GM-CSF polypeptides were constructed. These mutant polypeptides were designed based on a comparison of the human and murine GM-CSF polypeptide sequences and taking advantage of the specific binding displayed by 4K21 mAb for human GM-CSF.

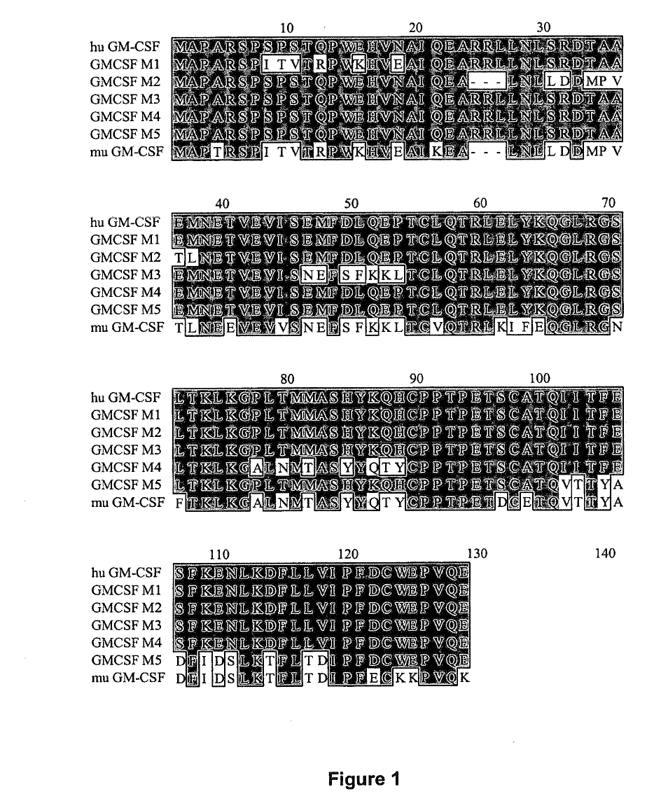

An alignment of the human and murine GM-CSF sequences (128 and 125 amino acids in length, respectively) using ClustalW revealed approximately 65% homology across the length of the sequences (see FIG. 1). Six mutant GM-CSF polypeptides were constructed (designated M1 to M5 and M5b) based on the wild type human sequence. In each mutant, amino acid substitutions were introduced in different regions of the molecule (boxed in FIG. 1) replacing human-derived sequences with sequences found at the corresponding location in the wild type murine sequence. The locations (with respect to the numbering of the human GM-CSF sequence as shown in FIG. 1 and SEQ ID NO:1) are as follows: M1, S8 to N18; M2, R24 to M37; M3, E46 to P53; M4, P77 to H88; M5, 1101 to V117 (SEQ ID NO:4); and M5b, S96 to N110 (SEQ ID NO:6).

Once designed, the M1 to M5 mutant polypeptide-encoding DNA sequences were optimised for expression in E. coli strain, BL21 (Geneart AG, Germany). For cloning into the bacterial expression vector pET28a (Novagen), NcoI (CCATGG) and HindIII (AAGCTT) sites were incorporated at the 5′ and 3′ ends respectively. A stop codon was incorporated before the HindIII site. The sequence of the wild type human GM-CSF sequence was purchased from ATCC (Catalog No. 39754). DNA was prepared from a single colony and the GM-CSF coding region amplified by PCR with NcoI and HindIII cloning sites using the following primers:

(SEQ ID NO: 9)
5′-CCGACCATGGCACCCGCCCGCTCGCCCAGCCCC-3′
forward primer;
and
(SEQ ID NO: 10)
5′-AAGCTTTCACTCCTGGACTGGCTCCCAGCAGTC-3′
reverse primer.

The amplified DNA was sequence verified and a difference (I101T) in the wild type sequence was observed when compared to that produced by a commercial supplier, Peprotech. The Peprotech sequence and that listed on the protein database (Accession No: NP_{—}000749) are identical and different to that in the ATCC clone. The different sequence in the ATCC clone was confirmed to originate from the template and hence may be a naturally occurring variant of GM-CSF as has been previously observed (Burgess et al., 1987, Blood, 69:43-51). This mutation at amino acid 101 was changed to the wild type sequence by site directed mutagenesis using the QuickChange® II XL (Stratagene) kit according to the manufacturer's instructions using the primers:

(SEQ ID NO: 11)
CTTGTGCAACCCAGATTATCACCTTTGAAAGTTTC forward primer;
and
(SEQ ID NO: 12)
GAAACTTTCAAAGGTGATAATCTGGGTTGCACAAG reverse primer.

To construct the hGMCSF M5b mutant, 3 sequential mutagenesis steps were performed using with the hGMCSF M5 mutant as the initial template. Each step was performed by site directed mutagenesis using the QuickChange® II XL (Stratagene) kit using different primers as follows:

hCSFM5a.f1
(SEQ ID NO: 13)
CCGACCCCGGAAACCGACTGTGAGACCCAGGTGACCACC
hCSFM5a.r2
(SEQ ID NO: 14)
GGTGGTCACCTGGGTCTCACAGTCGGTTTCCGGGGTCGG
hSCFM5b.f1
(SEQ ID NO: 15)
GATAGCCTGAAAACCTTTCTGCTCGTTATTCCGTTTGATTGCTGGG
hCSFM5b.r2
(SEQ ID NO: 16)
CCCAGCAATCAAACGGAATAACGAGCAGAAAGGTTTTCAGGCTATC
hCSFM5b.f3
(SEQ ID NO: 17)
GGATTTTATCGATAGCCTGAAAGACTTTCTGCTCGTTATTCCGTTTG
hCSFM5b.r4
(SEQ ID NO: 18)
CAAACGGAATAACGAGCAGAAAGTCTTTCAGGCTATCGATAAAATCC

Initially (step 1) primers hCSFM5a.f1 and hCSFM5a.r2 were used with hGMCSF M5 mutant template. Subsequently (step 2), primers hCSFM5b.f1 and hCSFM5b.r2 were used with the step 1 product, and finally (step 3) hCSFM5b.f3 and hCSFM5b.r4 primers were used with the step 2 product. A sequence verified clone was then subcloned into an expression vector, expressed, refolded and quantitated.

The DNA cloning process involved preparation of DNA by standard methods known to those skilled in the art: digestion of the plasmid DNA with NcoI and HindIII as recommended by the manufacturer (NEB); separation of DNA fragments by agarose gel electrophoresis; recovery of DNA fragments from the gel using a gel extraction kit (JetQuick, Genomed); ligation of gene fragment to vector fragment (T4 DNA ligase, NEB); and transformation of DNA into competent E. coli (TOP10, Invitogen). Plasmid DNA from transformed cells was analysed by restriction enzyme digest and the GM-CSF gene in the plasmid was sequenced to confirm that it was cloned in the correct reading frame with no mutations.

Plasmid DNA encoding the wild type or M1 to M5b mutant GM-CSF polypeptides were transformed into the E. coli BL21 (DE3) (Novagen) for expression. A single kanamycin resistant clone was isolated and incubated overnight at 37° C. in 5 ml of LB media with kanamycin (30 μg/ml). This starter culture was added to 500 ml of LB media and amplified at 37° C. for about 3 hrs until to OD600 was between 0.6 and 0.8. IPTG (Sigma, Cat #16758) was added to a final concentration of 400 μM and the cultures incubated for a further 4 hrs at 37° C. The bacteria were collected by centrifugation for 10 minutes at 10000 g, the supernatant discarded and the pellet weighed. The pellet was resuspended in Novagen Bugbuster (Novogen, Cat #71456) and inclusion bodies were isolated and purified as recommended by the manufacturer. The proteins in the inclusion bodies were resuspended in 6M urea and buffer exchanged using D-tubes Dialyzer Midi tubes in refolding buffer (20 mM sodium phosphate, 150 mM NaCl, 50 mM Tris HCl, pH 7.5). The protein concentration was quantitated by GM-CSF-specific ELISA.

### Example 3

### Binding of 4K21 to Human GM-CSF Mutants

The binding analysis of 4K21 to the mutant GM-CSF polypeptides M1 to M5b was performed using a semi-quantitative ELISA. Approximately equal amounts of GM-CSF were used to coat a Maxisorb (Nunc. Cat #464718) 384 well plate and serially diluted 2-fold to the bottom of the plate (16 times). Coating was performed overnight at room temperature. Plates were washed 3 times with wash buffer (1×PBS, 0.05% tween) and blocked for 1 hr at 37° C. with PBS, 1% BSA solution. Plates were then washed 3 times with wash buffer. 4K21 mAb (12.5 ng/ml) and a commercially available anti human GM-CSF antibody ((310 ng/ml) BD; Cat #554501) were loaded so that both antibodies gave equivalent binding with wt GMCSF. The plates were washed 3 times with wash buffer then secondary antibody, HRP conjugated goat anti mouse IgG (Jackson Cat #115-036-062) for 4K21 and rabbit anti rat IgG (BD, cat#P0450) for the BD commercial antibody was added to the wells at 1/5000 dilution and incubated at 37° C. for 1 hr. Plates were washed 4 times following which BD OptEIA TMB substrate (Cat #555214), prepared according to the manufacturer's instructions, was added to the wells and incubated at room temperature for 10 minutes. The reaction was stopped with 1M sulphuric acid and the plate read on a Tecan plate reader at 450 nm with reference filter at 620 nm.

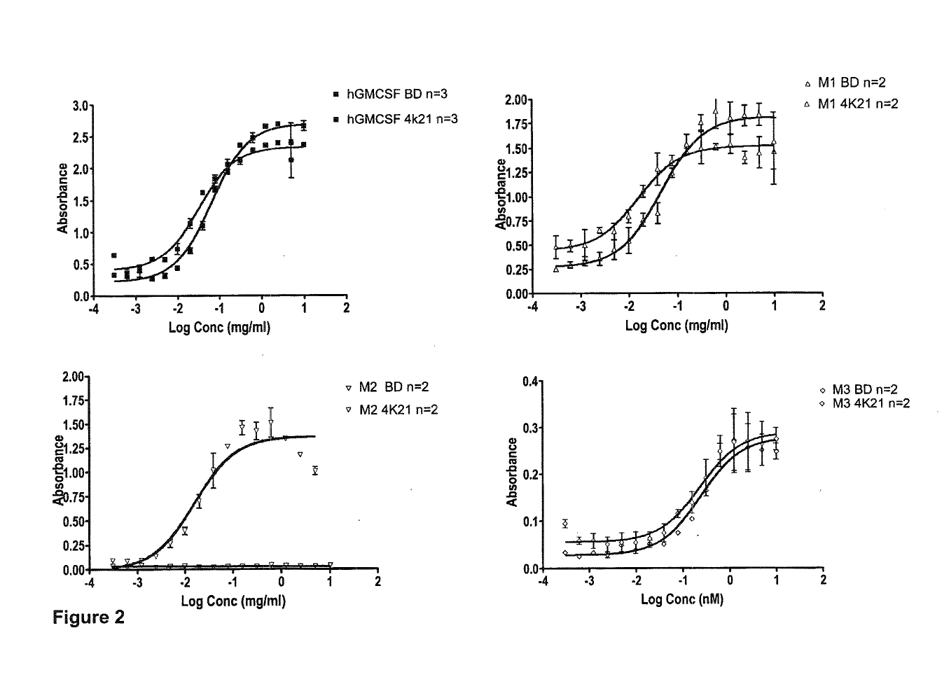

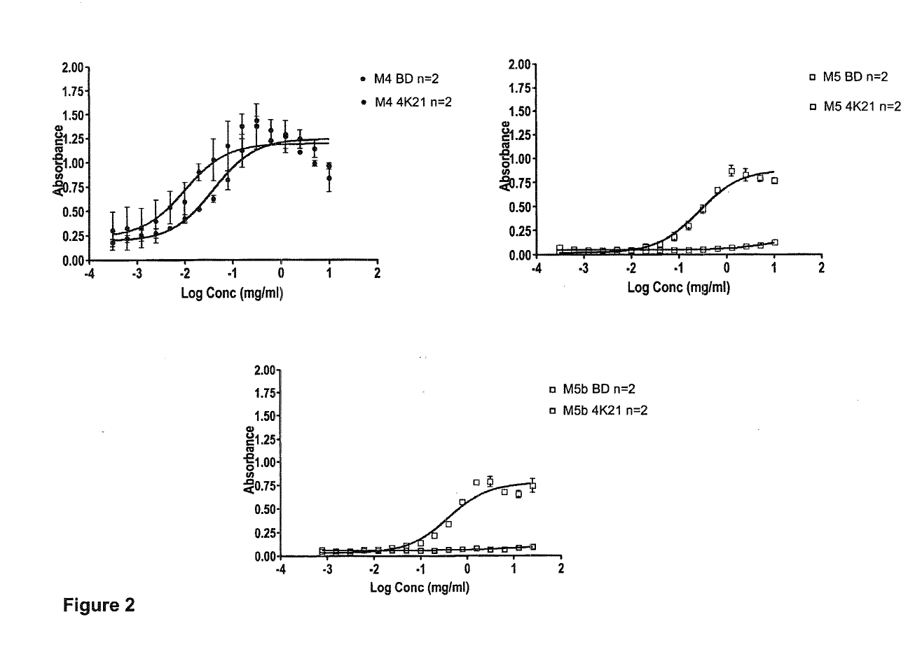

As shown in FIG. 2, both 4K21 mAb and a commercially available anti-human GMCSF antibody (BD; Cat #554501) bind wild type human GM-CSF. Binding to GM-CSF mutants M1, M3 and M4 is similar to that observed for wild type GM-CSF. Binding of 4K21 to mutant M3 is reduced (similar to the commercial BD antibody) and is absent for M5 and M5b. This indicates that 4K21 binds to the M5/M5b region of human GM-CSF. The commercially available BD anti human GM-CSF antibody was shown to bind to mutants M5 and M5b but not to mutant M2 indicating that it recognises a different epitope (FIG. 2).

The sequences of human and Macaque GM-CSF display very high sequence similarity with only one amino acid difference in the region mutated in the M5 and M5b mutants. Thus, the ability of the 4K21 mAb to bind wild type Macaque GM-CSF was also investigated. The results of a semi-quantitative ELISA (performed as detailed above) demonstrate that 4K21 mAb binds to Macaque GM-CSF (data not shown).

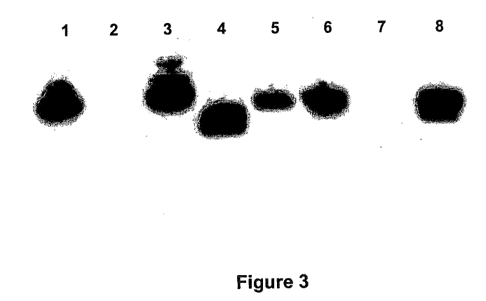

The binding of 4K21 mAb to the M5 region of human GM-CSF was confirmed by Western blot. Expression of the GM-CSF polypeptide was assessed semi-quantitatively using a Coomassie stained gel. Equal amounts of GM-CSF polypeptide was then separated by PAGE and transferred to nitrocellulose. The membrane was blocked with 5% skim milk powder and then probed with 4K21 mAb (1:1000, 2.68 ug/ml). The membrane was washed 3 times and then probed with a goat anti-mouse IgG-HRP (sc2005, Lot #H0505) diluted 1/5000. The membrane was washed 3 times and bound 4K21 was detected with Western Lightning Chemiluminescence reagent plus (Perkin Elmer, Cat #NEL105). FIG. 3 shows that 4K21 binds specifically to the wild type human GM-CSF, the GM-CSF mutants M1, M2, M3 and M4 and to Macaque GM-CSF. However 4K21 does not bind to GM-CSF mutant M5. A commercially available anti human GM-CSF antibody (BD: Cat #554501) was shown to bind mutant M5 but not to mutants M2 or M3 indicating that it recognises a different epitope (data not shown).

### Example 4

### Binding of 4K21 and a Humanized Form Thereof to Murine GM-CSF Mutants

To confirm the binding epitope for 4K21 mAb on human GM-CSF, the inventors constructed a series of GM-CSF mutant polypeptides in which the human-derived mutant regions defined above were inserted into a murine GM-CSF backbone. The ability of antibodies 4K21, hGM4/34 (humanized form of 4K21) and a commercially available rat anti-mouse GM-CSF antibody (BD; Cat #554403) were then tested using semi-quantitative ELISA. The BD rat anti-mouse GM-CSF antibody was used as a positive control. The 4K21 and hGM4/34 monoclonal antibodies do not bind murine GM-CSF (data not shown). The ability of a human-derived mutant region to restore binding of these monoclonal antibodies to a murine-based GM-CSF polypeptide is indicative of the importance of that mutant region to antibody binding.

Murine GMCSF mutants mGMCSF_hM3, mGMCSF_hM4, and mGMCSF_hM5 were constructed as described above for their human GM-CSF counterparts. For mGMCSF_hM5b, 4 sequential site-directed mutagenesis steps were performed with the mGMCSF hM5 as the initial template and using the following primers:

mCSFhM5a.f1
(SEQ ID NO: 19)
CCGCCGACCCCGGAAACCTCTTGCGCAACCCAGATTATTACC
mCSFhM5a.r2
(SEQ ID NO: 20)
GGTAATAATCTGGGITGCGCAAGAGGTTICCGGGGTC-GGCGG
mCSFhM5b.f1
(SEQ ID NO: 21)
CCTGAAAGATTTCCTGCTGGACATTCCGTTCGAATGCAAAAAAC
mCSFhM5b.r2
(SEQ ID NO: 22)
GTTTTTTGCATTCGAACGGAATGTCCAGCAGGAAATCTTTCAGG
mCSFhM5b.f3
(SEQ ID NO: 23)
GAAAACCTGAAAGATTTCCTGACGGACATTCCGTTCGAATGC
mCSFhM5b.r4
(SEQ ID NO: 24)
GCATTCGAACGGAATGTCCGTCAGGAAATCTTTCAGGTTTTC
mCSFhM5b.f5
(SEQ ID NO: 25)
CTTTCAAAGAAAACCTGAAAACTTTCCTGACGGACATTCCGTTCG
mCSFhM5b.r6
(SEQ ID NO: 26)
CGAACGGAATGTCCGTCAGGAAAGTTTTCAGGTTTTCTTTGAAAG

Initially (step 1) mCSFhM5a.f1 and mCSFhM5a.r2 primers were used with mGMCSF hM5 as template. Subsequently (step 2), mCSFhM5b.f1 and mCSFhM5b.r2 primers were used with the step 1 product, followed by (step 3) mCSFhM5b.f3 and mCSFhM5b.r4 primers with the step 2 product, and finally (step 4) mCSFhM5b.f5 and mCSFhM5b.r4 primers with the step 3 product. A sequence verified clone was then cloned into an expression vector, expressed, refolded and quantitated.

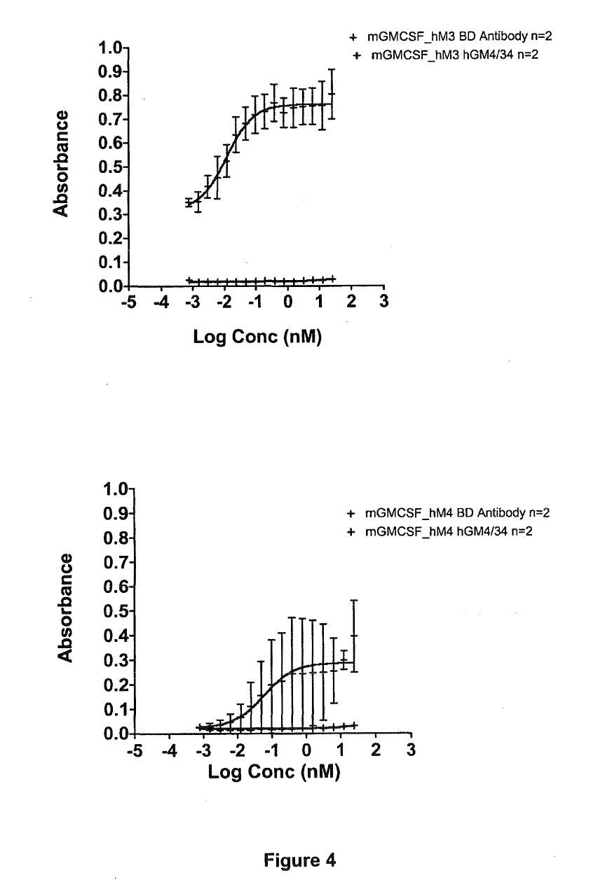

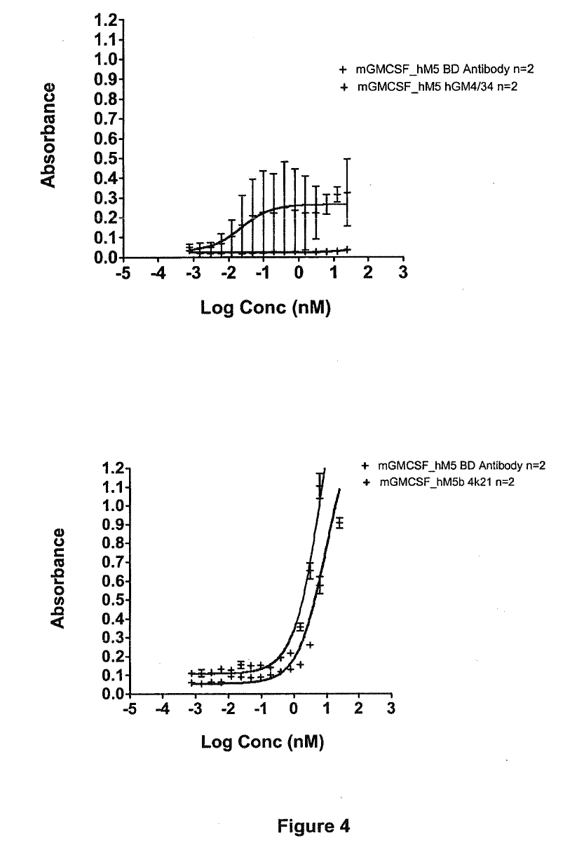

Binding analysis of the murine GMCSF mutants was performed using a similar procedure to that described above for the human counterparts, with exceptions based on the antibodies used. For the experiments depicted in FIG. 4, the rat anti-mouse GM-CSF BD antibody at 78 ng/ml was used to compare to either 4K21 at 12.5 ng/ml or hGM4/34 at 78 ng/ml. Secondary antibodies used were: polyclonal rabbit anti-rat Ig/HRP (Cat #P0450) at 1/2500 dilution for BD anti mouse GM-CSF BD antibody; goat anti-mouse IgG-HRP antibody (Cat #115-036-062) at 1/2500 dilution for 4K21 antibody; and mouse anti-human kappa-HRP (Cat #9220-05) at 1/10000 for hGM4/34 humanised antibody.

As shown in FIG. 4, the BD rat anti-mouse GM-CSF antibody bound to all murine GM-CSF mutant polypeptides. However humanized 4K21 mAb (hGM4/34) did not bind to either the mGMCSF_hM3, mGMCSF_hM4 or mGMCSF_hM5 polypeptides. In contrast 4K21 did bind to mGMCSF_hM5b. These results indicate that the region within the human GM-CSF molecule responsible for the binding of 4K21 (and human and humanized forms thereof) comprises the M5b region.

### Example 5

### Binding of 4K21 to Site-Directed (Alanine) Mutants of Human GM-CSF

To further define the GM-CSF epitope bound by 4K21, the inventors constructed a series of site-directed mutants within the human GM-CSF polypeptide sequence replacing wild type residues with alanine (A) residues. The ability of 4K21 to bind these mutants was then tested.

Site-directed mutagenesis was performed as described above. For the mutants C89A, S96A, C97A, T99A, Q100A and I101A, the following primers were used:

CSF C89A.f
(SEQ ID NO: 27)
CACTACAAGCAGCACGCCCCTCCAACCCCGGAAAC
CSF C89A.r
(SEQ ID NO: 28)
GTTTCCGGGGTTGGAGGGGCGTGCTGCTTGTAGTG
CSF S96A.f
(SEQ ID NO: 29)
CCAACCCCGGAAACTGCCTGTGCAACCCAG
CSF S96A.r
(SEQ ID NO: 30)
CTGGGTTGCACAGGCAGTTTCCGGGGTTGG
CSF C97A.f
(SEQ ID NO: 31)
CAACCCCGGAAACTTCCGCTGCAACCCAGATTATCACC
CSF C97A.r
(SEQ ID NO: 32)
GGTGATAATCTGGGTTGCAGCGGAAGTTTCCGGGGTTG
CSF T99A.f
(SEQ ID NO: 33)
GGAAACTTCCTGTGCAGCCCAGATTATCACCTTTGAAAG
CSF T99A.r
(SEQ ID NO: 34)
CTTTCAAAGGTGATAATCTGGGCTGCACAGGAAGTTTCC
CSF Q100A.f
(SEQ ID NO: 35)
CGGAAACTTCCTGTGCAACCGCGATTATCACCTTTGAAAGTTTC
CSF Q100A.r
(SEQ ID NO: 36)
GAAACTTTCAAAGGTGATAATCGCGGTTGCACAGGAAGTTTCCG
CSF I101A.f
(SEQ ID NO: 37)
CTTCCTGTGCAACCCAGGCTATCACCTTTGAAAGTTTC
CSF I101A.r
(SEQ ID NO: 38)
GAAACTTTCAAAGGTGATAGCCTGGGTTGCACAGGAAG

Sequence verified clones were then cloned into expression vectors, expressed, refolded and quantitated.

Binding analysis was carried out essentially as described above with the exception of the method of quantitation. The BD antibody was used to quantitate the relative expression of the alanine mutants as none of these mutants were in the M2 binding epitope of the BD antibody. Quantitation was performed with the BD rat anti-human GM-CSF antibody (Cat #554501) at 0.31 ug/ml with the secondary antibody being polyclonal rabbit anti-rat Ig/HRP (Cat # PO₄₅₀) at 1/2500 dilution. The binding was then compared to 4K21 used at 12.5 ng/ml with the secondary antibody being goat anti-mouse IgG-HRP antibody (Cat #115-036-062) at 1/2500 dilution.

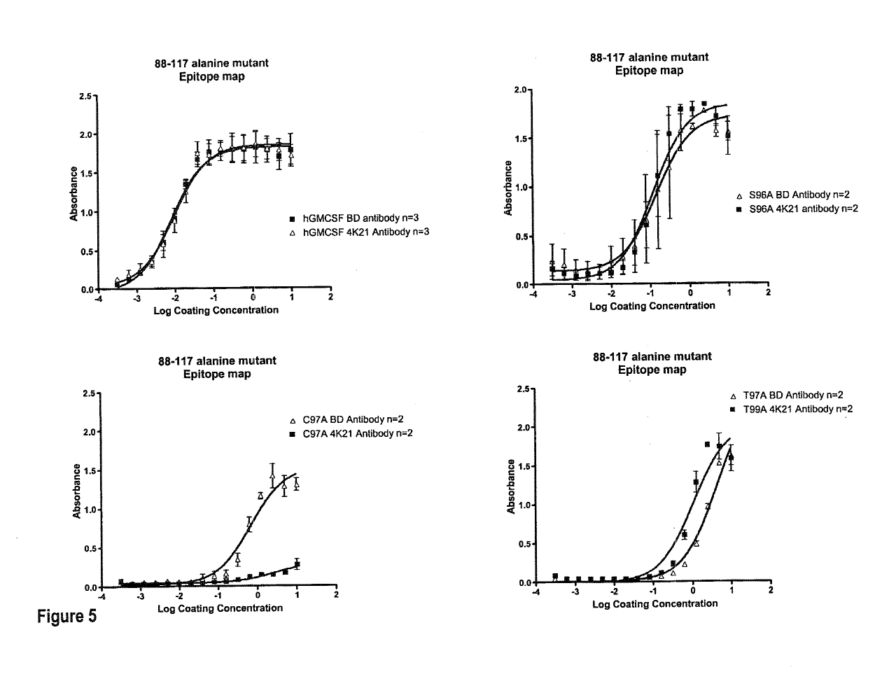

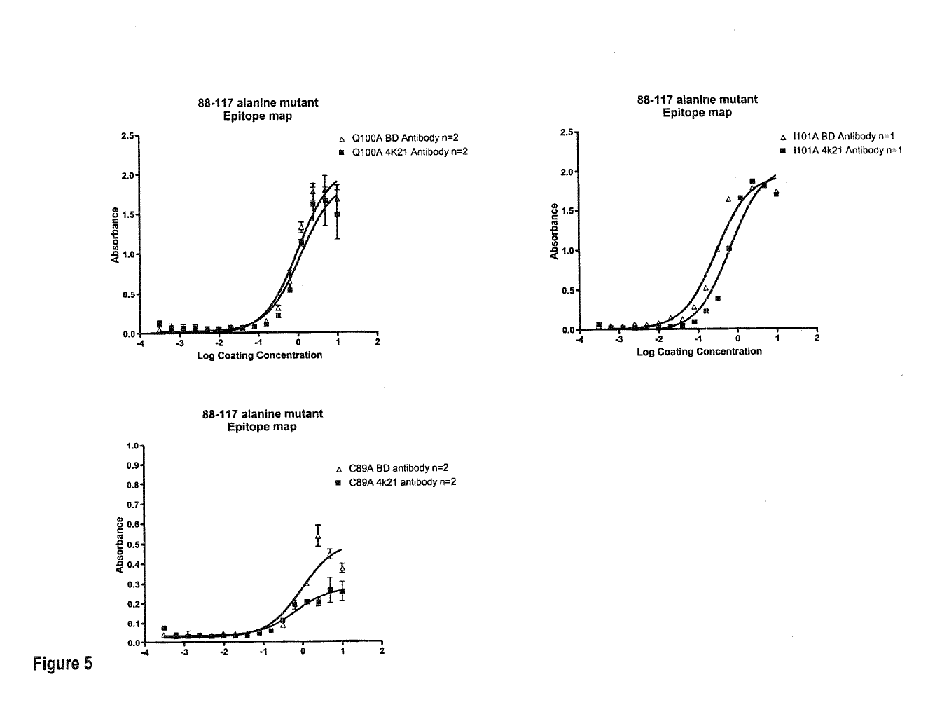

As shown in FIG. 5, mutations at residues C89 and 1101 to alanine resulted in modest reductions in the binding of 4K21, whereas mutation at residue C97 to alanine diminished 4K21 to almost negligible levels. Residue C97 of human GM-CSF therefore appears to be critical for binding of the 4K21 monoclonal antibody.

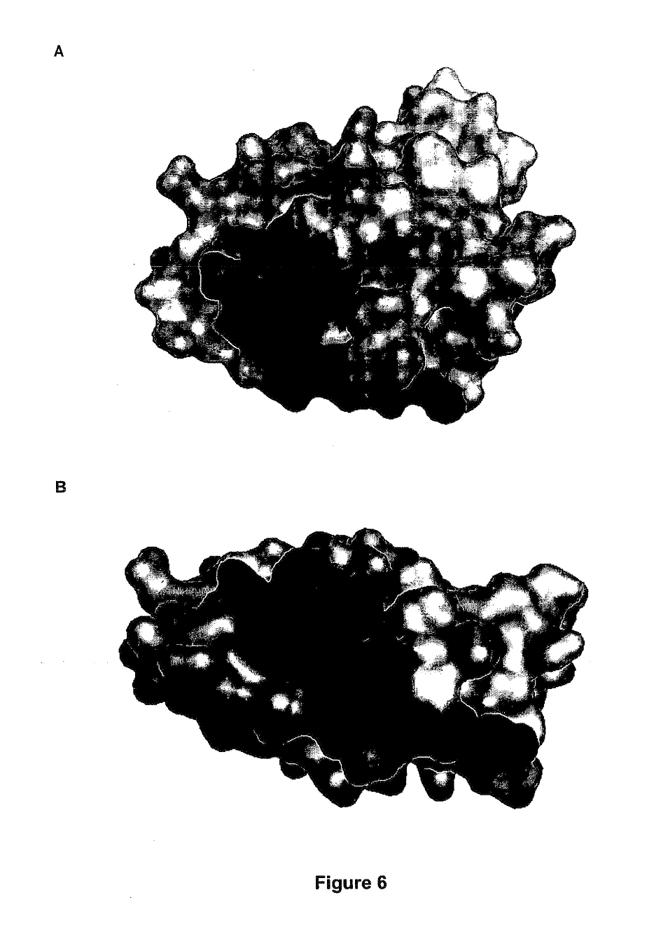

The above-described data in Examples 2 to 5 indicate that the region of human GM-CSF comprising the epitope recognised by neutralizing antibody 4K21 is located in the M5/M5b mutant regions. Thus, the minimum epitope appears to include the sequence SCATQIITFESFKEN (SEQ ID NO:6), located at residues 96 to 110 of the human GM-CSF sequence as set forth herein in SEQ ID NO:1. The crystal structure of human GM-CSF has been defined (Rozwarski et al., 1996 Proteins: Structure, Function and Genetics, 26:304-313). The empirically defined binding epitope for 4K21 on GM-CSF can be visualised with the Pymol program and shown to lie on the surface of the molecule (FIG. 6).

1. A compound or molecule that specifically interacts with or binds to a region of GM-CSF located between, or corresponding to a region located between, about residues 89 and 117 of the human GM-CSF polypeptide sequence as set forth in SEQ ID NO:1.

2. The compound or molecule of claim 1, wherein the molecule is an antibody.

3. The compound or molecule of claim 2, wherein said antibody specifically binds to a region of human GM-GSF that comprises an amino acid sequence set forth in any one of SEQ ID NOs:2 to 6, or a portion thereof.

4. The compound or molecule of claim 2 wherein said antibody specifically binds to a region of human GM-GSF that comprises the amino acid sequence set forth in SEQ ID NO:6, or a portion thereof.

5. The compound or molecule of claim 2, wherein said antibody is a neutralizing antibody capable of inhibiting the expression or activity of GM-CSF

6. The compound or molecule of claim 2, wherein said antibody is a polyclonal or monoclonal antibody.

7. The compound or molecule of claim 6, wherein said monoclonal antibody is the antibody designated mAb 4K21, or a humanized or human form thereof.

8. (canceled)

9. An immunogenic peptide consisting of an amino acid sequence set forth in any one of SEQ ID NOs :2 to 6, or a portion thereof.

10. The immunogenic peptide of claim 9, wherein said immunogenic peptide is a constituent of, or is derived from, human GM-CSF having the amino acid sequence set forth in SEQ ID NO:1.

11. (canceled)

12. An antibody that binds to an immunogenic peptide of claim 9.

13-16. (canceled)

17. A method for the treatment or prevention of a GM-CSF-mediated disease or condition or a disease or condition otherwise associated with elevated or aberrant GM-CSF expression or activity, the method comprising administering to a subject in need thereof an effective amount of: (i) an immunogenic peptide as defined in claim 9 to thereby generate an immune response against GM-CSF; or (ii) an antibody that binds to a region of human GM-CSF located between about residues 89 and 117 of the human GM-CSF polypeptide sequence as set forth in SEQ ID NO: 1 or to an immunogenic peptide as defined in claim 9.

18. (canceled)

19. The method of claim 17, wherein the disease or condition is an autoimmune or inflammatory disease or condition.

20. The method of claim 19, wherein the autoimmune or inflammatory disease or condition is selected from the group consisting of: asthma, rheumatoid arthritis, chronic obstructive pulmonary disease, idiopathic thrombocytopenic purpura, acute respiratory distress syndrome, multiple sclerosis, Alzheimer's disease, Crohn's disease, irritable bowel syndrome, colitis, psoriasis, macular degeneration, uveitis, Wallerian degeneration, antiphospholipid syndrome, restinosis, atherosclerosis, idiopathic pulmonary fibrosis, relapsing polychondritis, hepatitis, glomerulonephritis, lupus and other metabolic diseases.

21-22. (canceled)

23. The compound or molecule of claim 2, wherein said antibody specifically binds to a region of human GM-GSF that consists of an amino acid sequence set forth in any one of SEQ ID NOs:2 to 6, or a portion thereof.

24. The compound or molecule of claim 2 wherein said antibody specifically binds to a region of human GM-GSF that consists of the amino acid sequence set forth in SEQ ID NO:6, or a portion thereof.

25. The compound or molecule of claim 2 wherein said antibody has a variable light chain of SEQ ID NO: 7.


SEQUENCE LISTING (38 sequences)

SEQ ID NO: 1 - protein, 128 aa, Homo sapiens
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 2 - protein, 29 aa, Homo sapiens
CPPTPETSCATQIITFESFKENLKDFLLV

SEQ ID NO: 3 - protein, 22 aa, Homo sapiens
SCATQIITFESFKENLKDFLLV

SEQ ID NO: 4 - protein, 17 aa, Homo sapiens
IITFESFKENLKDFLLV

SEQ ID NO: 5 - protein, 22 aa, Homo sapiens
CPPTPETSCATQIITFESFKEN

SEQ ID NO: 6 - protein, 15 aa, Homo sapiens
SCATQIITFESFKEN

SEQ ID NO: 7 - protein, 112 aa, Mus musculus
DVVMTQTPLSLPVSLGDQASISCRSSQSLVNSNGNTYLHWFLQKPGQSPKLLIYKVSNRFSGVPDRFSGSGSGTDFTLKISRVEAEDLGVYFCSQSTHVPPTFGGGTKLEIK

SEQ ID NO: 8 - protein, 115 aa, Mus musculus
EVQLVESGGGLVKSGGSLKLSCAASGFAFSAYDMSWVRQTPEKRLELVAYISSGGSSFYYPDTVKGRFTISRDNAKNTLYLQMSSLKSEDTAMYYCTRHLGFDYWGQGTTLTVSS

SEQ ID NO: 9 - DNA, 33 bp, Artificial Sequence
  Synthetic sequence
ccgaccatggcacccgcccgctcgcccagcccc

SEQ ID NO: 10 - DNA, 33 bp, Artificial Sequence
  Synthetic sequence
aagctttcactcctggactggctcccagcagtc

SEQ ID NO: 11 - DNA, 35 bp, Artificial Sequence
  Synthetic sequence
cttgtgcaacccagattatcacctttgaaagtttc

SEQ ID NO: 12 - DNA, 35 bp, Artificial Sequence
  Synthetic sequence
gaaactttcaaaggtgataatctgggttgcacaag

SEQ ID NO: 13 - DNA, 39 bp, Artificial Sequence
  Synthetic sequence
ccgaccccggaaaccgactgtgagacccaggtgaccacc

SEQ ID NO: 14 - DNA, 39 bp, Artificial Sequence
  Synthetic sequence
ggtggtcacctgggtctcacagtcggtttccggggtcgg

SEQ ID NO: 15 - DNA, 46 bp, Artificial Sequence
  Synthetic sequence
gatagcctgaaaacctttctgctcgttattccgtttgattgctggg

SEQ ID NO: 16 - DNA, 46 bp, Artificial Sequence
  Synthetic sequence
cccagcaatcaaacggaataacgagcagaaaggttttcaggctatc

SEQ ID NO: 17 - DNA, 47 bp, Artificial Sequence
  Synthetic sequence
ggattttatcgatagcctgaaagactttctgctcgttattccgtttg

SEQ ID NO: 18 - DNA, 47 bp, Artificial Sequence
  Synthetic sequence
caaacggaataacgagcagaaagtctttcaggctatcgataaaatcc

SEQ ID NO: 19 - DNA, 42 bp, Artificial Sequence
  Synthetic sequence
ccgccgaccccggaaacctcttgcgcaacccagattattacc

SEQ ID NO: 20 - DNA, 42 bp, Artificial Sequence
  Synthetic sequence
ggtaataatctgggttgcgcaagaggtttccggggtcggcgg

SEQ ID NO: 21 - DNA, 44 bp, Artificial Sequence
  Synthetic sequence
cctgaaagatttcctgctggacattccgttcgaatgcaaaaaac

SEQ ID NO: 22 - DNA, 44 bp, Artificial Sequence
  Synthetic sequence
gttttttgcattcgaacggaatgtccagcaggaaatctttcagg

SEQ ID NO: 23 - DNA, 42 bp, Artificial Sequence
  Synthetic sequence
gaaaacctgaaagatttcctgacggacattccgttcgaatgc

SEQ ID NO: 24 - DNA, 42 bp, Artificial Sequence
  Synthetic sequence
gcattcgaacggaatgtccgtcaggaaatctttcaggttttc

SEQ ID NO: 25 - DNA, 45 bp, Artificial Sequence
  Synthetic sequence
ctttcaaagaaaacctgaaaactttcctgacggacattccgttcg

SEQ ID NO: 26 - DNA, 45 bp, Artificial Sequence
  Synthetic sequence
cgaacggaatgtccgtcaggaaagttttcaggttttctttgaaag

SEQ ID NO: 27 - DNA, 35 bp, Artificial Sequence
  Synthetic sequence
cactacaagcagcacgcccctccaaccccggaaac

SEQ ID NO: 28 - DNA, 35 bp, Artificial Sequence
  Synthetic sequence
gtttccggggttggaggggcgtgctgcttgtagtg

SEQ ID NO: 29 - DNA, 30 bp, Artificial Sequence
  Synthetic sequence
ccaaccccggaaactgcctgtgcaacccag

SEQ ID NO: 30 - DNA, 30 bp, Artificial Sequence
  Synthetic sequence
ctgggttgcacaggcagtttccggggttgg

SEQ ID NO: 31 - DNA, 38 bp, Artificial Sequence
  Synthetic sequence
caaccccggaaacttccgctgcaacccagattatcacc

SEQ ID NO: 32 - DNA, 38 bp, Artificial Sequence
  Synthetic sequence
ggtgataatctgggttgcagcggaagtttccggggttg

SEQ ID NO: 33 - DNA, 39 bp, Artificial Sequence
  Synthetic sequence
ggaaacttcctgtgcagcccagattatcacctttgaaag

SEQ ID NO: 34 - DNA, 39 bp, Artificial Sequence
  Synthetic sequence
ctttcaaaggtgataatctgggctgcacaggaagtttcc

SEQ ID NO: 35 - DNA, 44 bp, Artificial Sequence
  Synthetic sequence
cggaaacttcctgtgcaaccgcgattatcacctttgaaagtttc

SEQ ID NO: 36 - DNA, 44 bp, Artificial Sequence
  Synthetic sequence
gaaactttcaaaggtgataatcgcggttgcacaggaagtttccg

SEQ ID NO: 37 - DNA, 38 bp, Artificial Sequence
  Synthetic sequence
cttcctgtgcaacccaggctatcacctttgaaagtttc

SEQ ID NO: 38 - DNA, 38 bp, Artificial Sequence
  Synthetic sequence
gaaactttcaaaggtgatagcctgggttgcacaggaag